(19) United States
(12) Patent Application Publication
(10) Pub. No.: US 2024/0095912 A1
(43) Pub. Date: Mar. 21, 2024

(54) DEEP LEARNING-BASED ATTENUATION CORRECTION OF CARDIAC IMAGING DATA

(71) Applicant: CEDARS-SINAI MEDICAL CENTER, Los Angeles, CA (US)

(72) Inventors: Piotr SLOMKA, Los Angeles, CA (US); Aakash Shanbhag, San Pedro, CA (US)

(73) Assignee: CEDARS-SINAI MEDICAL CENTER, Los Angeles, CA (US)

(21) Appl. No.: 18/367,154

(22) Filed: Sep. 12, 2023

Related U.S. Application Data
(60) Provisional application No. 63/405,707, filed on Sep. 12, 2022.

(51) Int. Cl. G06T 7/00

(52) U.S. Cl. CPC G06T 7/0012; G06T 2207/10108; G06T 2207/20081; G06T 2207/30048

(57) ABSTRACT

Systems and methods are disclosed for applying attenuation correction to single photon emission computed tomography (SPECT) imaging data for myocardial perfusion imaging (MPI) studies. SPECT-MPI imaging data can be provided to a deep-learning model to automatically generate simulated computed tomography attenuation correction (CT-AC) images from the non-corrected (NC) SPECT-MPI imaging data. These simulated CT-AC images can then be used to perform attenuation correction on the SPECT-MPI imaging data to generate corrected SPECT-MPI imaging data. The deep-learning model can be trained using corresponding pairs of non-corrected SPECT-MPI imaging data and traditional CT-AC imaging data. The deep-learning model can be a conditional generative adversarial neural network (cGAN).

### CROSS-REFERENCE TO RELATED APPLICATIONS

The present application claims the benefit of U.S. Provisional Patent Application No. 63/405,707 filed Sep. 12, 2022 and entitled “DEEP LEARNING-BASED ATTENUATION CORRECTION OF CARDIAC IMAGING DATA,” which is hereby incorporated by reference in its entirety.

### FEDERALLY SPONSORED RESEARCH AND DEVELOPMENT

This invention was made with government support under Grant No. HL089765 awarded by the National Institutes of Health and Grant No. HL161195 awarded by the National Institutes of Health. The government has certain rights in the invention.

### TECHNICAL FIELD

The present disclosure relates to medical imaging generally and more specifically to post-processing of single photon emission computed tomography myocardial perfusion imaging data.

### BACKGROUND

Computed tomography (CT) medical imaging is an important tool used across many diagnostic and clinical settings. Specifically, single photon emission computed tomography (SPECT) myocardial perfusion imaging (MPI) is an especially useful tool used in the care of cardiac patients. SPECT involves detection of a radioisotope tracer that has been injected into a patient. SPECT MPI involves quantitatively assessing the perfusion of cardiac tissue, such as during a cardiac stress test. Abnormalities of regional perfusion can be used to identify patients with a higher likelihood of having obstructive coronary artery disease (CAD).

While SPECT MPI is a very useful tool, SPECT MPI images are susceptible to degradation (e.g., from surrounding soft tissue) that may affect the brightness or counts in the imaging data in fashion that is unrelated to tracer uptake or myocardial perfusion. Thus, attenuation correction (AC) has been developed as a methodology used to compensate for this type of degradation. AC improves the diagnostic accuracy and specificity of non-corrected (NC) SPECT-MPI images. Traditional AC techniques involve acquiring imaging data from a separate CT scan (e.g., x-ray-based CT scan) to develop a density map of the patient in question, which can then be used to correct the SPECT MPI imaging data.

Such traditional AC techniques can require dedicated, expensive combination SPECT/CT scanners, and the concomitant radiation exposure from the additional CT scanning. Such specialized SPECT/CT scanners are unavailable in many clinical settings, and thus AC is unavailable for patients at those locations. Further, when SPECT/CT scanners are available, traditional AC nevertheless involves subjecting the patient to additional ionizing radiation, which carries its own risks and may by contraindicated for some patients. Finally, even if SPECT/CT scanners are available and the patient is suitable for and willing to undergo the additional radiation exposure of the CT scan, traditional AC is nevertheless susceptible to misregistration errors of the separately acquired SPECT and traditional AC maps, which is often a source of artifacts that may diminish the clinical value of AC images. Thus, despite the advantages of AC, it is currently performed in only a minority of SPECT MPI scans, especially in the latest generation of solid-state scanners where traditional AC is performed in fewer than 5% of the sites.

Certain aspects of the present disclosure address these and other problems associated with attenuation correction in SPECT imaging data.

### SUMMARY

The term embodiment and like terms are intended to refer broadly to all of the subject matter of this disclosure and the claims below. Statements containing these terms should be understood not to limit the subject matter described herein or to limit the meaning or scope of the claims below. Embodiments of the present disclosure covered herein are defined by the claims below, supplemented by this summary. This summary is a high-level overview of various aspects of the disclosure and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this disclosure, any or all drawings and each claim.

Embodiments of the present disclosure include a method comprising receiving non-attenuation-corrected (NC) single photon emission computed tomography (SPECT) imaging data. The NC SPECT imaging data including a plurality of image slices. The method further comprising generating simulated attenuation-correction (AC) imaging data from the NC SPECT imaging data by applying the NC SPECT imaging data to a generator network of a conditional generative adversarial network (cGAN). The cGAN is trained using training data. The training data includes a plurality of NC SPECT training images and a corresponding plurality of traditional AC images. The method further comprises generating attenuation-corrected SPECT imaging data based at least in part on the NC SPECT imaging data and the generated simulated AC imaging data.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The specification makes reference to the following appended figures, in which use of like reference numerals in different figures is intended to illustrate like or analogous components.

FIG. 1 is a schematic diagram of depicting a computing environment for generating a deep learning model, according to certain aspects of the present disclosure.

FIG. 2 is a schematic diagram depicting a computing environment for acquiring and processing imaging data, according to certain aspects of the present disclosure.

FIG. 3 is a flowchart depicting a process for training a deep-learning model, according to certain aspects of the present disclosure.

FIG. 4 is a flowchart depicting a process for generating and using simulated AC SPECT imaging data, according to certain aspects of the present disclosure.

FIG. 5 is an image comparing non-corrected (NC), traditional attenuation correction (CT), and simulated attenuation correction (DeepAC) SPECT-MPI imaging data for different views of cardiac tissue according to certain aspects of the present disclosure.

FIG. 6 is a set of images depicting the differences between different types of attenuation correction of SPECT-MPI imaging data according to certain aspects of the present disclosure.

FIG. 7 is a chart depicting the comparison of absolute differences in Stress TPD for different types of attenuation correction according to certain aspects of the present disclosure.

FIG. 8 is a chart depicting the comparison of Stress TPD values for different types of attenuation correction according to certain aspects of the present disclosure.

FIG. 9 is a chart depicting the comparison of change analysis between different types of attenuation correction according to certain aspects of the present disclosure.

FIG. 10 is a schematic diagram depicting a model architecture for a deep learning model for generating simulated AC images according to certain aspects of the present disclosure.

FIG. 11 is a chart depicting diagnostic accuracy for obstructive coronary artery disease (CAD) for different types of SPECT-MPI data according to certain aspects of the present disclosure.

FIG. 12 is a chart depicting change analysis (e.g., voxel-by-voxel analysis after subtraction of co-registered images) for different types of SPECT-MPI data according to certain aspects of the present disclosure.

FIG. 13 is a chart depicting absolute difference in stress TPD for different types of SPECT-MPI data according to certain aspects of the present disclosure.

FIG. 14 is a combination image depicting multiple view of different types of SPECT-MPI data and positive change charts for a first example patient according to certain aspects of the present disclosure.

FIG. 15 is a combination image depicting multiple view of different types of SPECT-MPI data and positive change charts for a second example patient according to certain aspects of the present disclosure.

FIG. 16 is a combination image depicting multiple view of different types of SPECT-MPI data and positive change charts for a third example patient according to certain aspects of the present disclosure.

FIG. 17 is a combination image depicting multiple view of different types of SPECT-MPI data and positive change charts for a fourth example patient according to certain aspects of the present disclosure.

FIG. 18 is a combination image depicting multiple view of different types of SPECT-MPI data and positive change charts for a fifth example patient according to certain aspects of the present disclosure.

FIG. 19 is a block diagram of an example system architecture for implementing features and processes of the present disclosure.

### DETAILED DESCRIPTION

Certain aspects and features of the present disclosure relate to systems and methods for applying attenuation correction to single photon emission computed tomography (SPECT) imaging data for myocardial perfusion imaging (MPI) studies. SPECT-MPI imaging data can be provided to a deep-learning model to automatically generate simulated computed tomography attenuation correction (CT-AC) images from the non-corrected (NC) SPECT-MPI imaging data. These simulated CT-AC images can then be used to perform attenuation correction on the SPECT-MPI imaging data to generate corrected SPECT-MPI imaging data. The deep-learning model can be trained using corresponding pairs of non-corrected SPECT-MPI imaging data and traditional CT-AC imaging data. The deep-learning model can be a conditional generative adversarial neural network (cGAN).

To provide the benefits of traditional AC without problematic shortcomings, a deep learning (DL) model (DeepAC) was developed to apply AC directly to non-corrected (NC) images (e.g., NC short-axis images), without the use of CT (or the need of re-reconstruction of the data), by generating simulated AC images (DeepAC images). DeepAC is a cGAN comprised of two competing networks. A generator network is tasked with creating DeepAC images, while the discriminator network differentiates the DeepAC images from traditional AC images. The process is repeated until the discriminator network is no longer able to differentiate the traditional AC images from the DeepAC images.

By generating DeepAC SPECT images, physicians may be able to better evaluate images for potential artifacts and myocardial segmentation errors.

According to certain aspects of the present disclosure, simulated AC SPECT imaging data (e.g., short axis images) can be generated by a trained deep-learning neural network (e.g., a cGAN) that takes as input NC SPECT imaging data. These simulated AC SPECT images can thus be generated without the need for separate acquisition of CT imaging data. The simulated AC SPECT imaging data can be used to predict obstructive CAD through the calculation of total perfusion deficit (TPD) from the imaging data. Because the simulated AC SPECT imaging data is generated to be an accurate simulation of actual AC SPECT imaging data, the TPD calculations based on the simulated AC SPECT imaging data are similar to those when actual AC SPECT imaging data is used. Through external testing, it has been determined that the prediction performance for obstructive CAD is similar whether based on simulated AC SPECT imaging data or actual AC SPECT imaging data (p=0.196). Further, the simulated AC SPECT imaging data can additionally be used to generate derived polar maps for further analysis. In fact, review of simulated AC SPECT imaging data and/or derived polar maps may be useful to identify potential image artifacts during image interpretation.

In some cases, instead of or in addition to generating simulated AC SPECT imaging data, simulated AC polar maps can be generated by a trained deep-learning neural network (e.g., a cGAN) that takes as input NC polar maps (e.g., polar maps derived from NC SPECT imaging data). While simulated AC polar maps can be of some use, the prediction performance for obstructive CAD when using simulated AC polar maps is not much better than when predictions are made using NC SPECT imaging data (p=0.801). Overall, the prediction performance for obstructive CAD using actual (non-simulated) AC imaging data is significantly higher than when using NC imaging data. In fact, the prediction performance for obstructive CAD when using simulated AC SPECT imaging data (AUC 0.785, 95% CI 0.724-0.846) is significantly higher than when using simulated AC polar maps (AUC 0.720, 95% CI 0.651-0.790, p=0.021).

Certain aspects and features of the present disclosure improve how imaging analyzing computer systems operate by providing system for generating simulated AC imaging data accurately and without the need for additional CT imaging, thus allowing imaging data to be analyzed faster and with less radiation exposure to the patient.

Certain aspects and features of the present disclosure effect a particular treatment or prophylaxis for a disease or medical condition. For example, in some cases, certain aspects of the present disclosure can provide a neural network that can facilitate prediction of cardiac problem (e.g., obstructive CAD), which can provide a patient with advanced notice of when to begin treatment or take other actions to protect against major cardiac events, faster than existing techniques and without the additional radiation exposure normally required for attenuation correction.

Certain aspects and features of the present disclosure are closely integrated in and provide significant benefits to a specific field of technology, such as the interpretation of cardiac SPECT imaging data to predict obstructive CAD. While techniques have been used in the past to interpret SPECT imaging data, certain aspects and features of the present disclosure allow for NC imaging data to be leveraged with the improved accuracy and benefits of AC imaging data without the need for acquiring separate CT imaging data.

In a first example study, SPECT-MPI imaging was performed using Tc-99m sestamibi or Tc-99m tetrofosmin on scanners with solid-state multi-pinhole detectors. A cGAN was developed to generate simulated attenuation-corrected images (DeepAC). The model of this example study was trained using 798 (train 700: validation 98) pairs of non-corrected and CT-AC MPI studies performed at a single site.

The model was tested using studies from an external dataset (n=178). The agreement of various measures obtained automatically via DeepAC with those obtained via standard AC was assessed using quantitative clinical software. These measures included stress total perfusion deficit (S-TPD) and stress volume (S-VOL), as well as perfusion change for AC vs. DeepAC and NC scans. Wilcoxon rank-sum test was used to compare median values of S-TPD and perfusion change.

The median (IQR) of S-TPD was 4.54 (1.39, 11.29) for AC and 5 5.50 (2.02, 11.69) for DeepAC scans (p=0.4). The mean difference in S-TPD was 0.32 (95% Confidence Interval [CI]: −0.06, 0.71) between AC and DeepAC and −1.15 (95% CI: −1.66, −0.64) between AC and NC. The median S-VOL was 76.45 (54.35, 103.04) for AC and 76.43 (53.31, 105.44) DeepAC (p>0.9). The median change (IQR) was 11.33% (7.31, 17.75) and 2.25 (1.07, 4.10) for AC vs. NC and AC vs. DeepAC and, respectively (p<0.001). Thus, automatic clinical measurements of stress TPD and myocardial volume do not differ significantly between DeepAC and AC MPI scans. Perfusion change values as measured by the clinical software are significantly smaller between AC vs. DeepAC than AC vs. NC scans.

These illustrative examples are given to introduce the reader to the general subject matter discussed here and are not intended to limit the scope of the disclosed concepts. The following sections describe various additional features and examples with reference to the drawings in which like numerals indicate like elements, and directional descriptions are used to describe the illustrative embodiments but, like the illustrative embodiments, should not be used to limit the present disclosure. The elements included in the illustrations herein may not be drawn to scale.

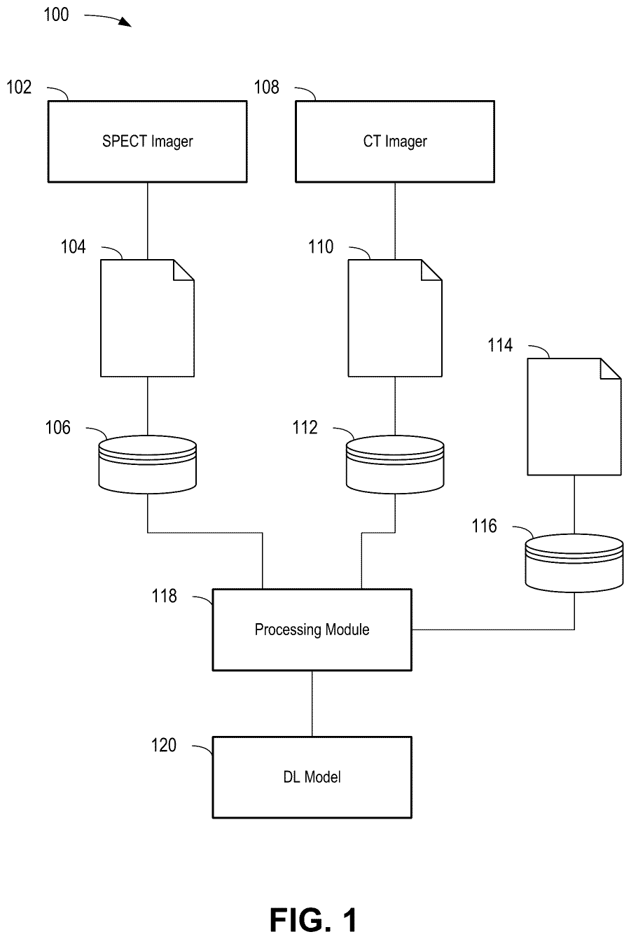

FIG. 1 is a schematic diagram of depicting a computing environment 100 for generating a deep learning model, according to certain aspects of the present disclosure. The deep learning model can be used to generate AC SPECT imaging data from NC SPECT imaging data. As used herein, SPECT imaging data can refer to cardiac SPECT imaging data.

A SPECT imager 102 can generate NC SPECT imaging data 104. The NC SPECT imaging data can include a set of image slices acquired using the SPECT imager 102, which can represent the presence or absences of radiotracer detected in the subject (e.g., patient) at different locations in the subject, such as by a gamma camera. The NC SPECT imaging data can be formatted and/or stored in any suitable format, including as 2D imaging data or 3D volumetric data. The NC SPECT imaging data 104 can be stored in a NC SPECT database 106.

A CT imager 108 can generate CT imaging data 110. The CT imaging data 110 can include a set of image slices acquired using a CT imager 108, which can represent the reflection and/or absorption, by tissue in a subject, of x-ray radiation directed at the subject at different locations in the subject. The CT imaging data can be formatted and/or stored in any suitable format, including as 2D imaging data or 3D volumetric data.

NC SPECT imaging data 104 and CT imaging data 108 can be provided to a processing module 118 (e.g., one or more computers or other suitable data processing apparatuses) to output AC SPECT imaging data 114, which can be stored in an AC SPECT database 116. The processing module 118 can generate a density map of a subject using the CT imaging data 108 and then apply that map to the NC SPECT imaging data 104 of the same subject to perform attenuation correction, thus resulting in AC SPECT imaging data 114. While described with reference to a CT imager 104 and CT imaging data 108, any suitable technique can be used to generate a density map of the subject and then use that density map to generate the AC SPECT imaging data 114 from the NC SPECT imaging data 104. The NC SPECT imaging data 104 and the AC SPECT imaging data 114 can be used as training data to train the DL network 120.

The processing module 118 (e.g., the same processing module that generated the AC SPECT imaging data 114 or another processing module) can use the NC SPECT imaging data 104 and the AC SPECT imaging data 114 to train a DL model 120. The DL model 120 can be a conditional generative adversarial network, including a generator and a discriminator. The generator can generate simulated AC SPECT imaging data from the NC SPECT imaging data 104, while the discriminator can work to differentiate the simulated AC SPECT imaging data from the actual AC SPECT imaging data 114. The generator and discriminator can be trained until the discriminator is no longer able to sufficiently differentiate the simulated AC SPECT imaging data from the actual AC SPECT imaging data 114.

The computing environment 100 can be implemented in a single location or across many locations. For example, many SPECT imagers 102 and CT imagers 108 from locations all over the world can be used to generate respective imaging data, which can be stored in disparate databases present in different locations.

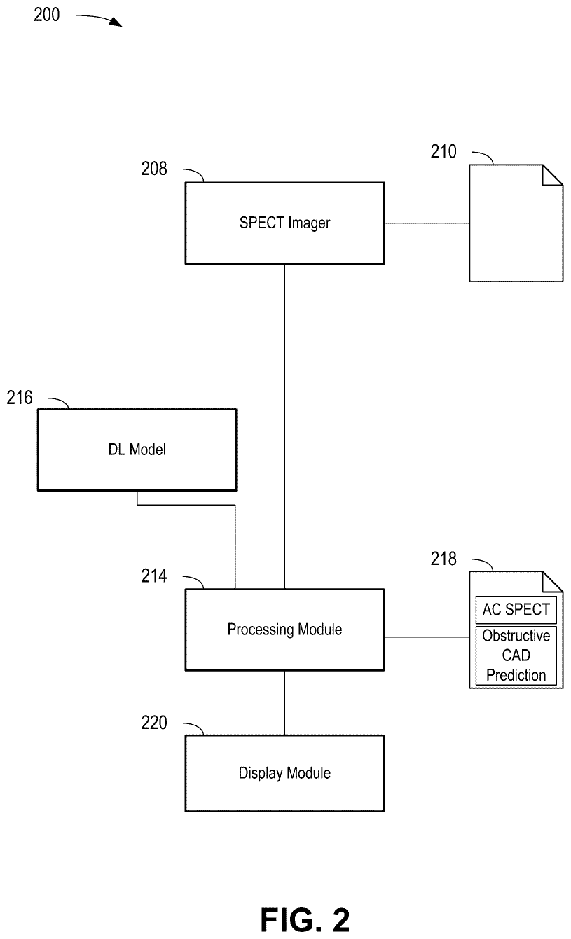

FIG. 2 is a schematic diagram depicting a computing environment 200 for acquiring and processing imaging data, according to certain aspects of the present disclosure. The computing environment 200 can include a SPECT imager 208, a processing module 214, and a display module 220. The SPECT imager 208, processing module 214, and display module 220 can be incorporated into a single housing or split into any number of housings, whether physically coupled together or not. The SPECT imager 208, processing module 214, and display module 220 can be located in a shared location (e.g., a room, suite, facility, or building) or in different locations. In some cases, the SPECT imager 208 can be located in a first location and the processing module 214 and display module 220 can be located in a separate, second location. For example, the SPECT imager 208 can be a SPECT imaging machine located in a medical imaging facility and the processing module 214 and display module 220 can be a physician's computer workstation (e.g., the processor and display of the computer workstation) in the physician's office that is located in a separate facility, separate city, or even separate county as the medical imaging facility. Other combinations can occur.

The SPECT imager 208 can be any suitable imaging device for generating nuclear imaging data 210 of cardiac tissue. The SPECT imager 208 can include detector(s) and any other components usable to generate and/or transmit the NC SPECT imaging data 210 from the data acquired by the detector(s). The SPECT imager 208 can be used to detect emissions resulting from a radiotracer, such as a gamma-emitting radioisotope.

The SPECT imager 208 can be communicatively coupled to the processing module 214 and/or the display module 220 via any suitable technique, such as wired or wireless connections, including direct connections or networked connections. In some cases, SPECT imager 208 can be coupled to processing module 214 via a network, such as a local area network, a wide area network, a cloud network, or the Internet. In some cases, data transfer between the SPECT imager 208 and the processing module 214 can occur via removable physical media, such as compact disks or flash drives.

The NC SPECT imaging data 210 can be stored and/or transferred in any suitable format. In some cases, the NC SPECT imaging data 210 can be stored and/or displayed as two-dimensional or three-dimensional images. In some cases, the NC SPECT imaging data 210 can be stored as a collection of data points or voxels.

The processing module 214 can be any suitable computing device for processing the NC SPECT imaging data 210 as disclosed herein. The processing module 214 can receive the imaging data 210 and analyze the NC SPECT imaging data 210 to generate output data 218. The output data 218 can include simulated AC SPECT imaging data, derived data from simulated AC SPECT imaging data (e.g., polar maps), an obstructive CAD prediction, and/or other usable predictions or data.

The processing module 214 can make use of a trained DL model 216 (e.g., DL model 120 of FIG. 1) to generate AC SPECT imaging data from the NC SPECT imaging data 210. The processing module 214 can output the AC SPECT imaging data as output data 218, and/or can further process the AC SPECT imaging data to generate further data (e.g., polar maps, TPD calculations, obstructive CAD predictions, and the like).

In some cases, the processing module 214 can include an input device, such as a computer mouse, keyboard, touchscreen, or the like. The input device can allow a user (e.g., a physician or other medical professional) to interact with the NC SPECT imaging data 210 and control generation of the output data 218. In some cases, the processing module 214 can include the display module 220 for displaying NC SPECT imaging data 210 and/or output data 218. In some cases, the display module 220 is used in conjunction with or includes an input device.

The output data 218, once generated, can be presented on the display module 220 or otherwise presented to a user or patient. The output data 218, especially an obstructive CAD prediction, can be usable to help tailor a treatment plan for a patient.

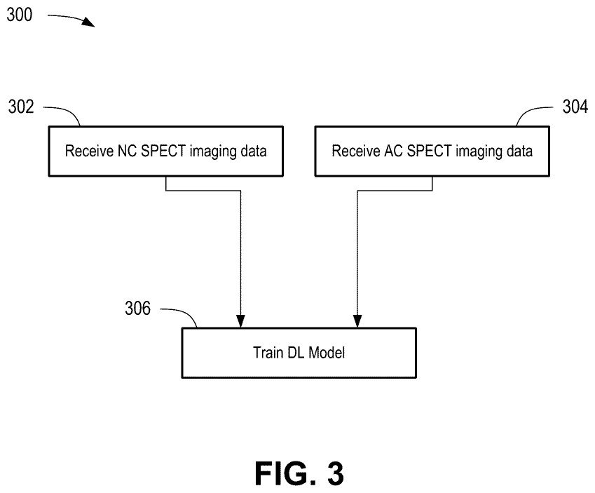

FIG. 3 is a flowchart depicting a process 300 for training a deep-learning model, according to certain aspects of the present disclosure. Process 300 can be performed using any suitable hardware, such as processing module 118 of FIG. 1.

At block 302, NC SPECT imaging data is received. Receiving NC SPECT imaging data can include receiving sets of NC SPECT short-axis images. Each set can pertain to a SPECT imaging study. In some cases, each set can pertain to a unique individual.

At block 304, AC SPECT imaging data is received. The AC SPECT imaging data can be attenuation corrected SPECT imaging data (e.g., SPECT short-axis images) derived from the NC SPECT imaging data from block 302 through an attenuation correction process. The AC SPECT imaging data can be correlated with the NC SPECT imaging data, such that each NC SPECT image can be associated with a correlated AC SPECT image based on that NC SPECT image.

The AC SPECT imaging data can be attenuation corrected by applying a density map to the NC SPECT imaging data. In some cases, the density map can be generated from analysis of related CT imaging data.

In some cases, receiving the AC SPECT imaging data at block 304 further includes generating the AC SPECT imaging data. Generating the AC SPECT imaging data can include obtaining a density map associated with the NC SPECT imaging data and applying that density map to the NC SPECT imaging data to account for attenuation present in the NC SPECT imaging data, thus resulting in AC SPECT imaging data. In some cases, the density map can be obtained through analysis of secondary imaging data, such as imaging data from a CT imager. This secondary imaging data (e.g., CT imaging data) can be registered with the NC SPECT imaging data and analyzed to generate a density map that is registered with the NC SPECT imaging data.

At block 306, the DL model is trained. Training the DL model includes providing the received NC SPECT imaging data from block 302 and the AC SPECT imaging data from block 304 to the DL model. Training the DL model at block 306 can include supplying the NC SPECT imaging data to a generator network of a cGAN to generate simulated AC SPECT imaging data. Training the DL model at block 306 can further include supplying the AC SPECT imaging data from block 304 and the simulated AC SPECT imaging data to a discriminator network of a cGAN to generate discriminator output (e.g., classifications of whether or not each of the inputs is actual or simulated imaging data). Training the DL model can include updating weights associated with layers of the generator network and/or discriminator network until the generator network is able to generate simulated AC SPECT imaging data from NC SPECT imaging data that is sufficiently indistinguishable, to the discriminator network, from actual AC SPECT imaging data. In some cases, a cost function of the cGAN is based on an absolute error measured between each simulated AC SPECT image and its corresponding AC SPECT image.

In some cases, training the DL model at block 306 can include supplying to the cGAN a region of interest for each set of NC SPECT imaging data and its corresponding AC SPECT imaging data. In some cases, this region of interest can be centered on a left ventricle within the NC SPECT imaging data.

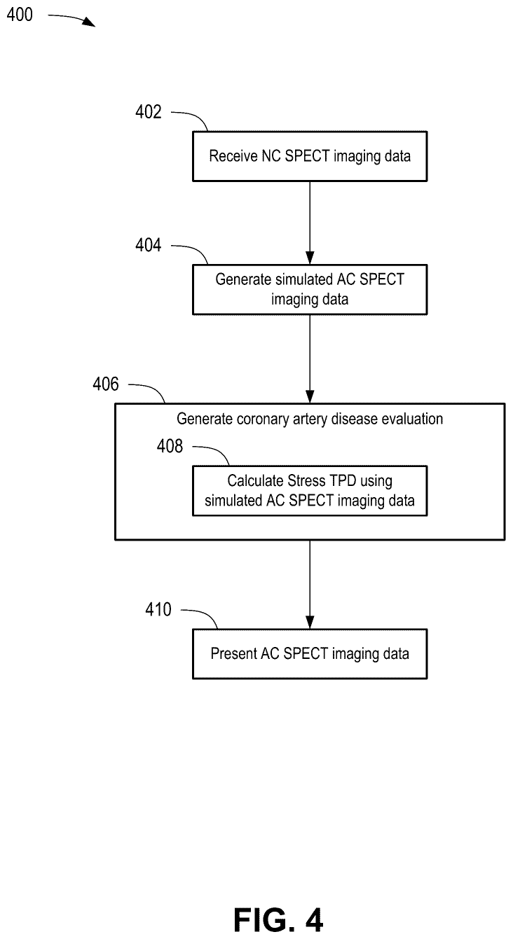

FIG. 4 is a flowchart depicting a process 400 for generating and using simulated AC SPECT imaging data, according to certain aspects of the present disclosure. Process 400 can be performed using any suitable hardware, such as processing module 214 of FIG. 2.

At block 402, NC SPECT imaging data can be received. The NC SPECT imaging data received at block 402 can be acquired of a particular subject (e.g., a patient).

At block 404, simulated AC SPECT imaging data is generated based on the NC SPECT imaging data received at block 402. Generating the simulated AC SPECT imaging data can include applying the NC SPECT imaging data to a trained DL model (e.g., a trained generator network of a cGAN), such as a DL model trained as described with reference to process 300 of FIG. 3.

At block 406, a coronary artery disease evaluation can be generated based on the simulated AC SPECT from block 404. The coronary artery disease evaluation can be any suitable metric indicative of or usable to evaluate a subject's risk associated with a coronary artery disease. For example, generating a coronary artery disease evaluation at block 406 can include calculating a Stress TPD using the simulated AC SPCET imaging data at block 408. The Stress TPD calculated at block 408 can be used to make a prediction of or diagnosis of the subject experiencing obstructive CAD. In some cases, the coronary artery disease evaluation includes generating a diagnosis of a coronary artery disease, such as obstructive CAD.

In some optional cases, at block 406, the simulated AC SPECT imaging data can be presented. Simulated AC SPECT imaging data can be presented on a display device or any other suitable output device. In some cases, presenting the simulated AC SPECT imaging data can include generating and/or presenting derivative data, such as polar maps derived from the simulated AC SPECT imaging data.

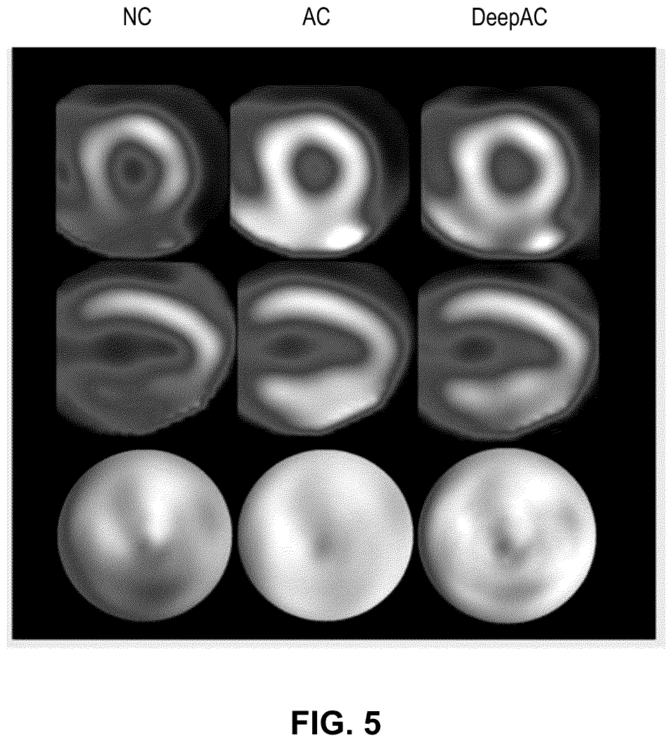

FIG. 5 is an image comparing non-corrected (NC), traditional attenuation correction (CT), and simulated attenuation correction (DeepAC) SPECT-MPI imaging data for different views of cardiac tissue according to certain aspects of the present disclosure. As seen in FIG. 5, DeepAC can achieve very similar results to that of traditional AC. DeepAC can be performed with a SPECT-only imager and does not require the patient to be subjected to additional doses of radiation needed for traditional AC.

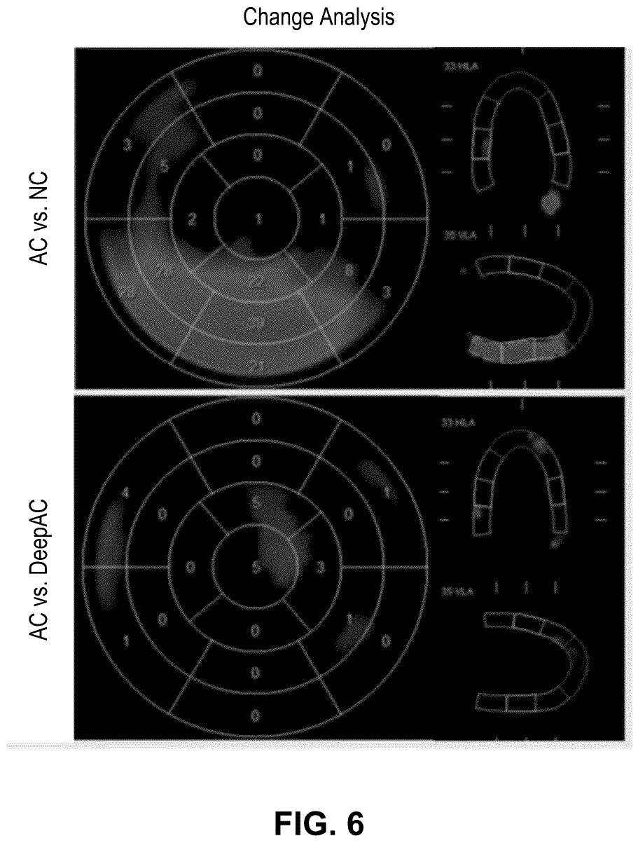

FIG. 6 is a set of images depicting the differences between different types of attenuation correction of SPECT-MPI imaging data according to certain aspects of the present disclosure. As seen in FIG. 6, the differences between non-corrected SPECT-MPI imaging data and SPECT-MPI imaging data that has been corrected using traditional AC are substantial, as expected. Also as seen in FIG. 6, the differences between SPECT-MPI imaging data corrected using traditional AC and corrected using Psuudo-AC are minimal. DeepAC is a suitable replacement for traditional AC.

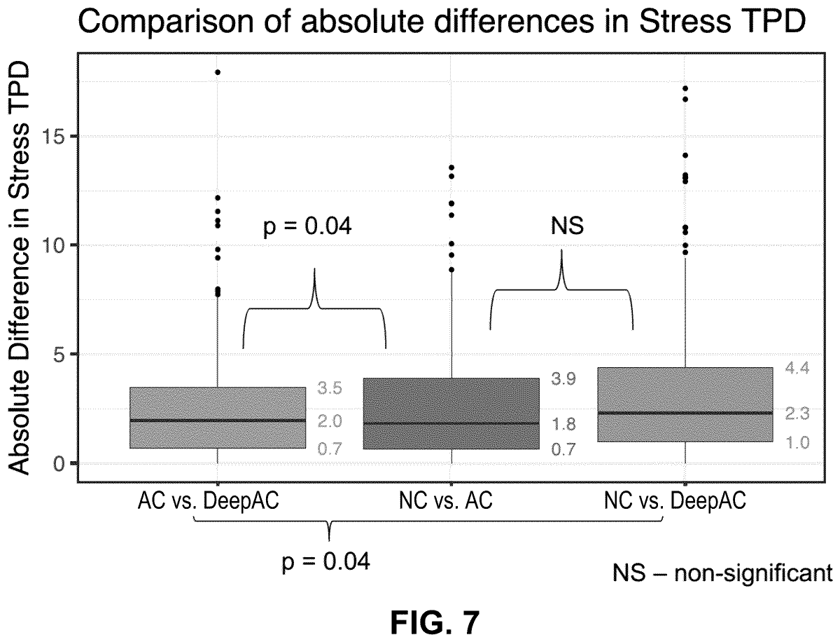

FIG. 7 is a chart depicting the comparison of absolute differences in Stress TPD for different types of attenuation correction according to certain aspects of the present disclosure.

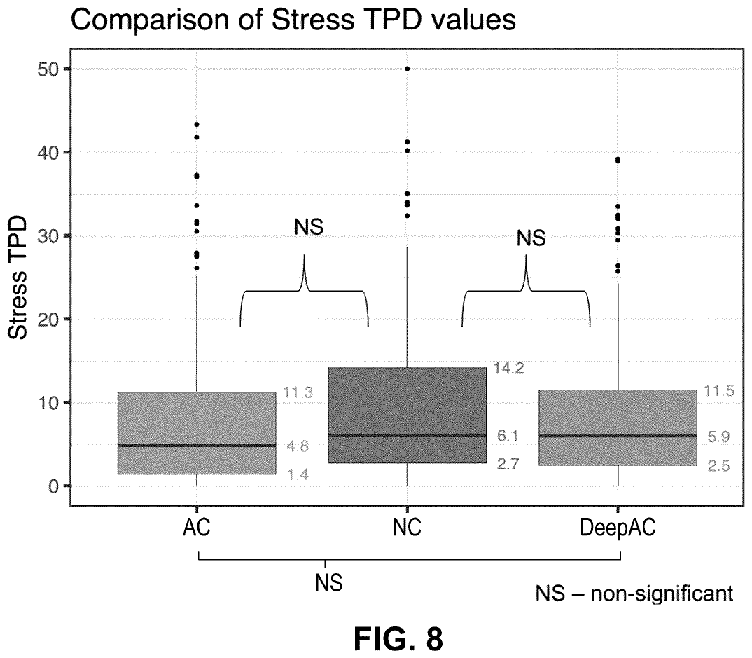

FIG. 8 is a chart depicting the comparison of Stress TPD values for different types of attenuation correction according to certain aspects of the present disclosure.

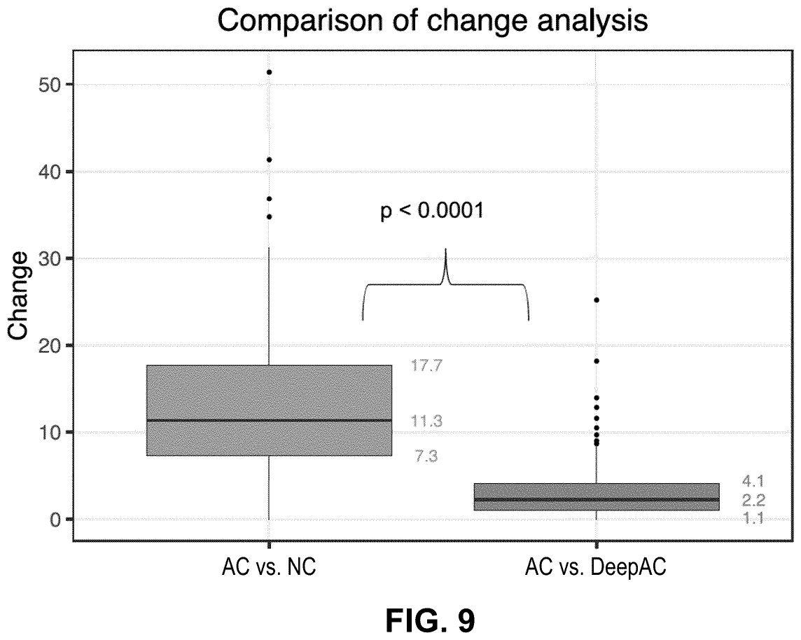

FIG. 9 is a chart depicting the comparison of change analysis between different types of attenuation correction according to certain aspects of the present disclosure. The comparison depicted in FIG. 9 can be the same changes depicted in FIG. 6.

In a second example study, image quantification using non-attenuation corrected (NC) images to CT-based (traditional) AC and simulated (DeepAC) images was compared. In an independent external dataset, diagnostic accuracy of quantitative perfusion analysis for obstructive CAD was compared for DeepAC, NC, and traditional AC imaging. A change analysis was performed to better understand the potential improvement in DeepAC images compared to traditional AC images.

In the second example study, SPECT-MPI imaging was performed using Tc-99m sestamibi or Tc-99m tetrofosmin on contemporary scanners with solid-state detectors. A cGAN was established to generate the DeepAC images. The model was trained with short-axis NC and AC images performed in one site (n=4886) and was tested in patients from two separate external sites (n=604). Diagnostic accuracy of stress total perfusion deficit (TPD) obtained from NC, AC, and DeepAC images for obstructive coronary artery disease (CAD) was assessed with area under the receiver operating characteristic curve (AUC). Direct count change between AC, NC, and DeepAC images on a per-voxel basis was also quantified.

DeepAC could be obtained in <1 second from NC images, AUC for obstructive CAD was higher for DeepAC TPD (0.79, 95% CI 0.72-0.85) compared to NC TPD (0.70, 95% Confidence Intervals (CI) 0.63-0.78, p<0.001), and similar to AC TPD (0.81, 95% CI 0.75-0.87, p=0.196). The normalcy rate (defined as stress TPD<5%) in the low likelihood population was higher for DeepAC TPD (84.0%) and AC TPD (84.6%) compared to NC TPD (64.5%, p<0.001 for both). Positive count change (increase in counts) was significantly higher for AC vs NC (median 9.4, Inter Quartile Range (IQR) 6.0-14.2, p<0.001) than for AC vs DeepAC (median 2.4, interquartile range [IQR] 1.3-4.2).

It was determined that DeepAC provides improved diagnostic accuracy for obstructive CAD similar to actual AC, as compared to NC images. DeepAC simplifies the task of artifact identification for physicians, avoids misregistration artifacts, and can be performed rapidly without the need for CT hardware and additional acquisitions.

In this second example study, two separate populations from separate centers were included. The model was trained with 4886 patients (45% female) from a single center who underwent SPECT MPI with CTAC. The model was then tested in an external population of 604 patients (48% male) from two different centers.

All scans were performed per SPECT/CT MPI guidelines. In the training population, patients underwent imaging using 99m Tc-tetrofosmin with a Discovery 570c scanner (GE Healthcare, Haifa, Israel). In the external testing populations, patients underwent either a 99m Tc-Sestamibi rest-stress or a 99m Tc-tetrofosmin stress-rest protocol with a Discovery 570c scanner (GE Healthcare, Haifa, Israel). Weight-adjusted mean (±standard deviation [SD]) doses of 403±207 MBq (10.9±5.6 mCi) were used in the training population and 512±157 MBq (13.8±4.2 mCi) for the external population. Stress images were acquired 15-60 minutes after stress over a total of 4-6 minutes. Patients underwent exercise or pharmacologic stress using standard clinical parameters.

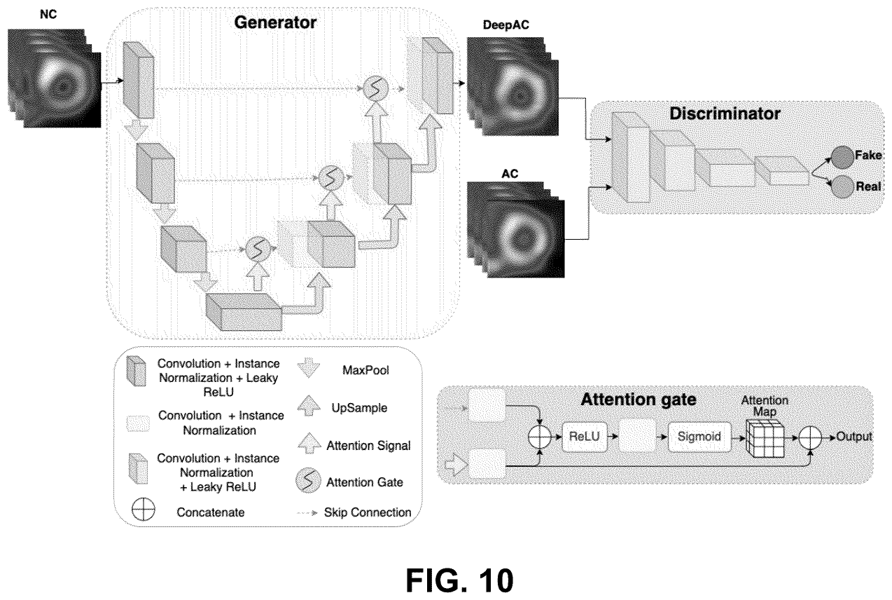

FIG. 10 is a schematic diagram depicting a model architecture for a deep learning model for generating simulated AC images according to certain aspects of the present disclosure. The model is a conditional generator adversarial network. First, the generator network creates simulated AC images from non-corrected (NC) images. The discriminator is then tasked with differentiating actual (traditional) AC images (“real”) from the simulated, DeepAC images (“fake”). The generator is an attention-gated 3D UNet where maximum pooling (MaxPool) downsamples the features. The attention gate takes input from the lower level (attention signal) with the skipped connection; it includes a rectified linear unit (ReLU) as a non-linear activation and generates an attention map which is concatenated to the upsampled level. This arrangement helps the generator network focus on essential image structures.

The model was developed using 4886 (train 4398: validation 488) pairs of NC and AC short axis SPECT slices from stress acquisitions from aa single site. In some cases, the method focuses on CT-free direct estimate of SPECT AC generation and is independent of any imaging information from CT. Ground truth short-axis SPECT AC images (reconstructed at 4×4×4 mm with slice thickness of 4 mm) were used to compare DeepAC.

Batch mode was used for model testing. Using a graphics processing unit (GeForce RTX 2080), the mean time to generate DeepAC images volume from AC volume was 9 milliseconds. Using a computer similar to a standard reporting workstation (AMD Ryzen 9 5950X 16-Core Processor, 64 Gb RAM), the mean inference time was 66 milliseconds.

Quantitative image comparisons were performed in the external testing population. Stress TPD was quantified using traditional methods and dedicated normal limit databases with Quantitative Perfusion SPECT (QPS) software (Cedars-Sinai Medical Center, Los Angeles, CA). Quantification of TPD for DeepAC images was performed using existing sex-specific databases for AC studies. Additionally, a “change” analysis was implemented (e.g., as implemented in clinical QPS software) to perform voxel-by-voxel comparisons between AC images, NC images, and DeepAC images The “change” analysis allows derivation of positive and negative count change between image pairs (sum of absolute voxel-by-voxel count changes in both directions) without normal databases. Positive change integrates image voxels with an increase in counts on AC imaging and negative change integrates voxels where AC image has decreased counts compared to NC image. Thus, positive change identifies perfusion defects which are corrected by the reference technique and negative change identifies relative perfusion defects unmasked by the reference technique. Change analysis can be used clinically to detect subtle differences in image sets; for example, when comparing stress and rest images, it could be used to identify areas of ischemia

Diagnostic accuracy was assessed in patients with same-day SPECT and coronary CT angiography (n=280) and low likelihood of coronary disease (LLK) (n=324). One population of patients underwent coronary CT angiography on the same day as SPECT MPI. Obstructive CAD was defined as any stenosis≥70% or ≥50% in the left main coronary artery. To ensure the prevalence of obstructive CAD is similar to that seen in a suspected CAD referral cohort, the population was enriched with a LLK population from another population. The LLK population included patients who did not undergo revascularization within 90 days of SPECT MPI and met the following criteria: 1) low-probability of CAD based on the Diamond-Forrester model, 2) normal expert visual interpretation of perfusion, 3) coronary artery calcium (CAC) score of 0, and 4) left ventricular ejection fraction>50%.

Standard descriptive statistics were used. The Morgan-Pitman test was used to compare variance between the differences of AC and DeepAC and AC and NC data. Diagnostic accuracy of obstructive CAD was assessed using area under the receiver operating characteristic curve (AUC). DeLong's test was used to evaluate for differences in AUC. Normalcy rates in the LLK population were evaluated, with normal quantitative perfusion defined as stress TPD<5%. Statistical tests were two-sided with p-value<0.05 considered significant.

Obstructive CAD was present in 64/604 (10.6%) patients in the external testing population compared to 10.7% in a large randomized controlled trial.

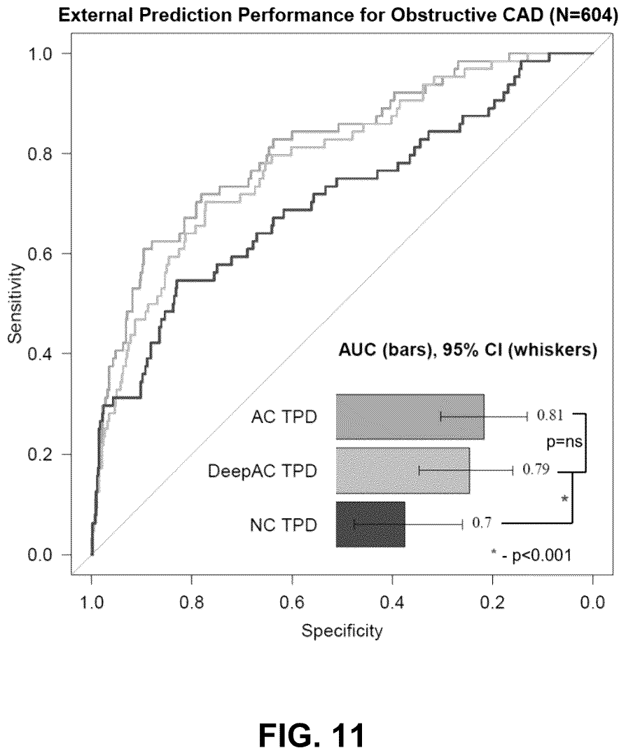

FIG. 11 is a chart depicting diagnostic accuracy for obstructive coronary artery disease (CAD) for different types of SPECT-MPI data according to certain aspects of the present disclosure. The area under the receiver operating characteristic curve (AUC) for AC (blue) and DeepAC (grey) stress total perfusion deficit (TPD) was higher compared to non-attenuation corrected (NC) stress TPD (red) There was no significant (ns) difference between DeepAC stress TPD and AC stress TPD (blue).

The AUC for DeepAC stress TPD (AUC 0.79, 95% CI 0.62-0.85) was higher compared to NC TPD (0.70, 95% CI 0.63-0.78, p<0.001). There was no difference in the AUC of DeepAC TPD compared to AC TPD (AUC 0.81, 95% CI 0.725-0.87, p=0.196). At 80% sensitivity, the specificity of DeepAC TPD was 64%, compared to 65% for AC TPD and 36% for NC TPD. The normalcy rate (defined as stress TPD<5%) in the LLK population was higher for DeepAC TPD (84.0%) and AC TPD (84.6%) compared to NC TPD (64.5%, p<0.001 for both).

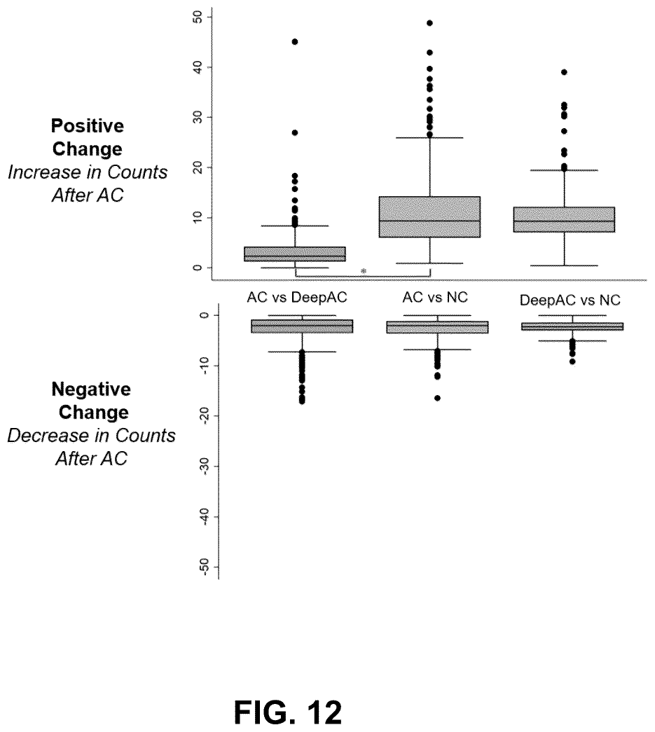

FIG. 12 is a chart depicting change analysis (e.g., voxel-by-voxel analysis after subtraction of co-registered images) for different types of SPECT-MPI data according to certain aspects of the present disclosure. Traditional attenuation corrected (AC) images were the reference image for comparisons of AC vs DeepAC (red) and AC vs non-attenuation corrected (NC) images (green). DeepAC images were used as the reference for DeepAC vs NC (blue). Negative change was not significantly different across all comparisons. However, positive change was significantly lower for AC vs DeepAC compared to AC vs non-attenuation corrected images (* p<0.001).

Positive change was significantly lower, representing closer agreement, for AC vs DeepAC (median 2.4, IQR 1.3-4.2) compared to AC vs NC (median 9.4, IQR 6.0-14.2, p<0.001). However, negative change was similar for AC vs DeepAC (median 2.0, IQR 0.9-3.5) compared to AC vs NC (median 2.0, IQR 1.2-3.6, p=0.935). Absolute differences between AC TPD vs DeepAC stress TPD were lower compared to the absolute differences between AC stress TPD and NC stress TPD (median 1.2 vs 2.3, p<0.001) (FIG. 4) Limits of agreement for AC TPD vs DeepAC TPD (bias −0.2, 95% limits of agreement −6.5 to 6.1; Spearman's Rho 0.78) were closer compared AC TPD vs NC TPD (bias −1.0, 95% limits of agreement −8.7 to 6.7; Spearman's Rho 0.55; p<0.001).

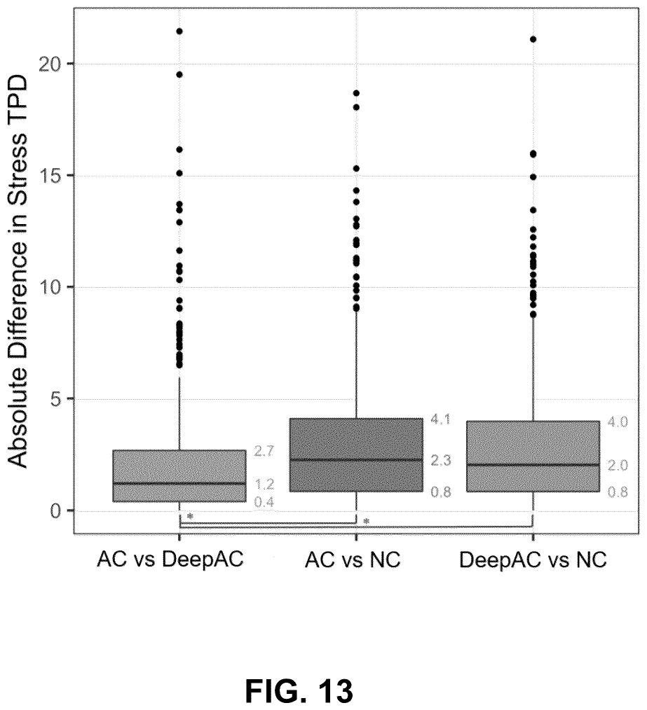

FIG. 13 is a chart depicting absolute difference in stress TPD for different types of SPECT-MPI data according to certain aspects of the present disclosure. Absolute differences in stress total perfusion deficit (TPD) between attenuation corrected (AC), DeepAC, and non-attenuation corrected (NC) values. Median absolute difference was lower for AC vs DeepAC (red), compared to AC vs NC (green) or DeepAC vs NC (blue) (both p<0.001).

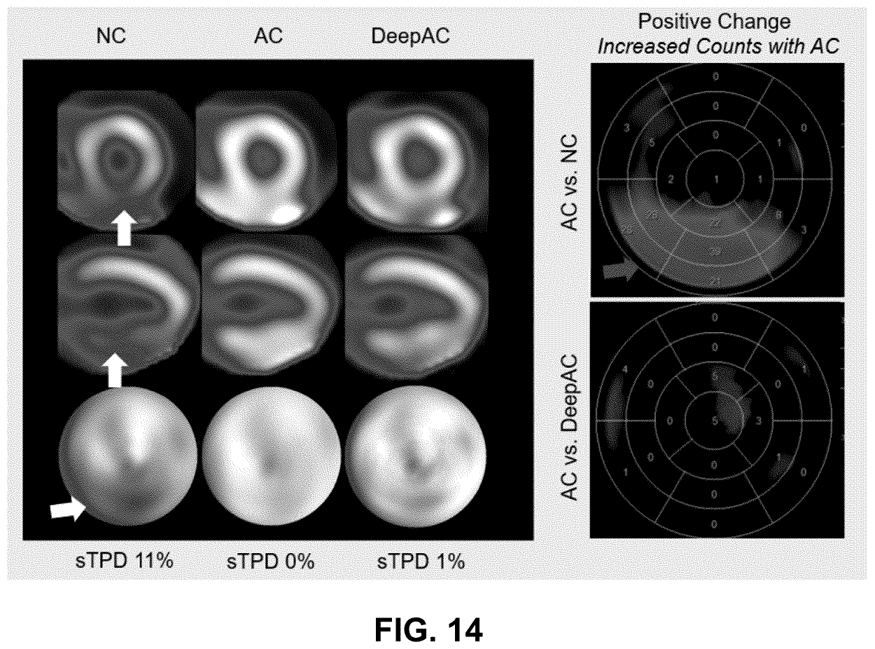

FIG. 14 is a combination image depicting multiple view of different types of SPECT-MPI data and positive change charts for a first example patient according to certain aspects of the present disclosure. The combination image includes non-attenuation corrected (NC), attenuation corrected (AC) and DeepAC images from a 53-year-old man with body mass index of 36. On short-axis images (top), vertical long axis (middle), and polar maps (bottom) there was a defect in the inferior wall on NC images only (white arrows), with evidence of adjacent radiotracer activity in the abdomen. Standard quantification by stress total perfusion deficit (sTPD) was 11% (abnormal). After AC correction, the sTPD was 0%, DeepAC correction resulted in sTPD 1% (both normal). There was a positive change of inferior wall counts for AC vs NC (red arrow). There was no change seen between AC vs DeepAC images. The patient had no coronary artery disease on coronary computed tomography angiography and the defect most likely represents diaphragmatic attenuation.

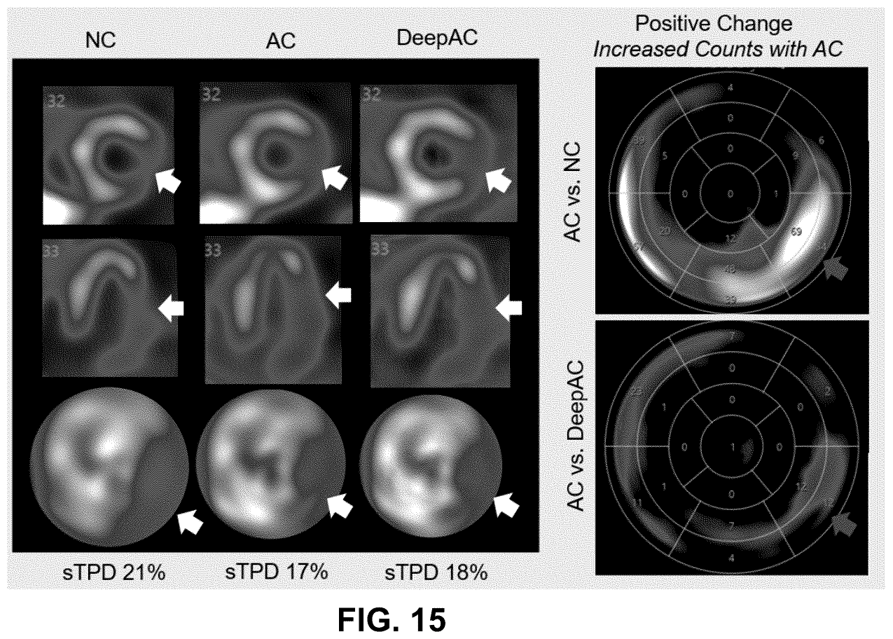

FIG. 15 is a combination image depicting multiple view of different types of SPECT-MPI data and positive change charts for a second example patient according to certain aspects of the present disclosure. FIG. 15 depicts non-attenuation corrected (NC), attenuation corrected (AC) and DeepAC images from a 63-year-old woman with body mass index of 32. On short-axis images (top), vertical long axis (middle) and polar maps (bottom) there was a defect in the anterolateral, inferolateral, and inferior walls on NC images (white arrows). Standard quantification by stress total perfusion deficit (sTPD) was 21% (abnormal). After AC correction, the sTPD was 17%, DeepAC correction resulted in sTPD 18% (both abnormal). There was positive change in the inferior and inferoseptal walls for AC vs NC (red arrows). There was a only a small area of positive change in the inferolateral wall for AC vs DeepAC. The patient had an 80% stenosis of the proximal left circumflex.

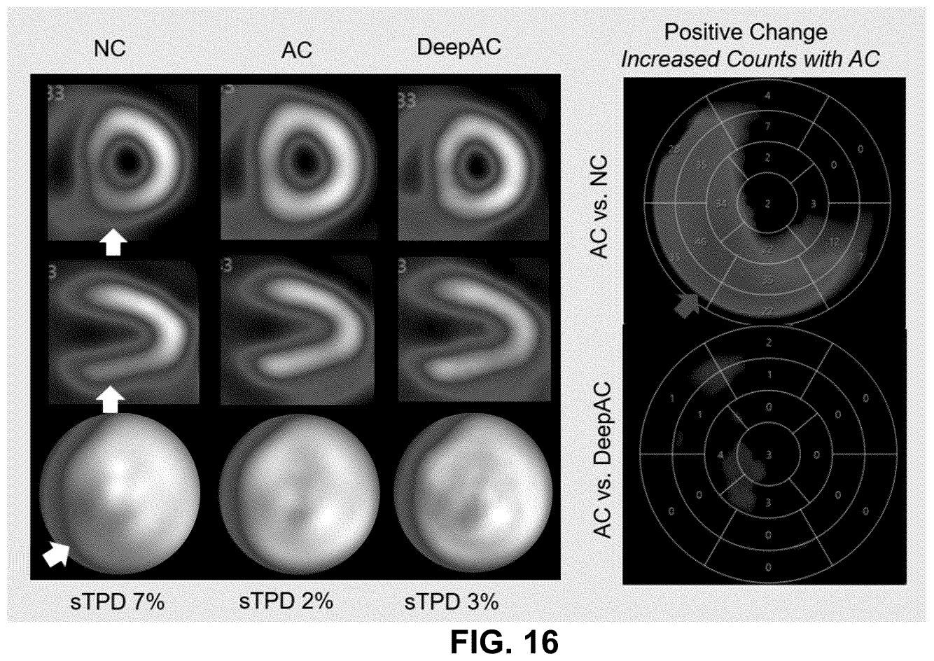

FIG. 16 is a combination image depicting multiple view of different types of SPECT-MPI data and positive change charts for a third example patient according to certain aspects of the present disclosure. FIG. 16 depicts non-attenuation corrected (NC), attenuation corrected (AC) and DeepAC images from a 62-year-old woman with body mass index of 29. On short-axis images (top), vertical long axis (middle) and polar maps (bottom) there was a defect in the inferior and inferoseptal walls on NC images only (white arrows). Standard quantification by stress total perfusion deficit (sTPD) was 7% (abnormal). After AC correction, the sTPD was 2%, DeepAC correction resulted in sTPD 3% (both normal). There was a positive change of inferior and inferoseptal wall counts for AC vs NC (red arrow). There was no change seen between AC vs DeepAC images. The patient had no coronary artery disease on coronary computed tomography angiography.

Certain aspects and features of the present disclosure make use of a deep learning model based on cGAN approach which directly generates DeepAC images, without traditional CT AC, from NC image sets. The DeepAC generates simulated AC SPECT images directly from NC images. It eliminates the possibility of CT misregistration, leverages optimized vendor-specific reconstruction algorithms for dedicated collimators and solid-state scanners, and allows physicians to evaluate full image sets for potential artifacts using the same approaches they would for any other clinical study. The model generates DeepAC images in a fraction of a second on standard computer hardware and could readily be implemented in clinical workflows as an automatic pre-processing step.

The diagnostic accuracy of DeepAC was higher compared to NC, as seen when applied to a large, external testing population. Additionally, using clinical quantitative analysis, DeepAC images were demonstrated to be more similar to traditional AC images as compared to NC images. DeepAC approach could be applied clinically in laboratories without dedicated CTAC hardware, which are a majority of SPECT-MPI laboratories, to increase normalcy rates and diagnostic accuracy, without affecting existing imaging protocols.

Conditional generative adversarial networks can be especially useful for generating simulated AC images. Other techniques to generate simulated AC SPECT-MPI imaging data, such as some non-conditional GANs, dual squeeze and excitation residual dense networks, and certain convolutional networks may not provide as effective results. Further, using NC images as input data can be especially useful for generating simulated AC imaging data, rather than other approaches, such as using NC polar maps as input data.

According to certain aspects of the present disclosure, the diagnostic accuracy obtained with DeepAC was higher than with NC images, and the specificity was approximately 30% higher, as identified when applied to a large external testing population from two different sites using standard clinical quantification of SPECT MPI. Also demonstrated was improved similarity between traditional AC and DeepAC SPECT images as compared to NC images.

Certain aspects of the present disclosure can be applied in various fashions. A model as disclosed herein can generate simulated SPECT short-axis images, rather than corrected polar maps. This approach allows physicians to identify potential sources of artifact such as excessive gut activity and potential errors in myocardial contours. It was not necessary to exclude cases with surface mismatch or segmentation errors as may have been necessary in other polar map-based approaches. The approach according to certain aspects of the present disclosure further leverages existing vendor-specific reconstruction algorithms and can be implemented as a pre-processing step prior to interpretation with any SPECT MPI interpretation software. Further, in an independent population, it was demonstrated that DeepAC improved diagnostic accuracy for obstructive CAD and normalcy rates, compared to NC SPECT. A very conservative definition of low-likelihood, including CAC score of 0, was used to minimize any chance of misclassifying presence of obstructive CAD in the absence of defined coronary anatomy.

Certain aspects of the present disclosure could be applied clinically to correct for soft-tissue attenuation without the additional radiation exposure associated with CT. An algorithm as described herein could be applied to correct NC SPECT MPI in sub-second times, avoiding potential issues with image misregistration. An algorithm as described herein could also be used at sites which typically rely on two-position imaging for soft-tissue AC, in the subset of patients who are unable to complete prone or upright imaging. The significant difference in cost and additional physical space required for a dedicated hybrid SPECT/CT camera system precludes their use in many nuclear medicine laboratories. Additionally, an algorithm according to the present disclosure could be combined with stress first imaging to provide significantly higher normalcy rate compared to NC images. Machine learning can be used for identifying patients for rest scan cancellation with a low probability of obstructive CAD or with a low risk of major adverse cardiovascular events. The quantitative perfusion information from DeepAC datasets could be used to further improve the accuracy of these algorithms.

In some cases, further improvements in diagnostic accuracy may be achieved by applying dedicated DeepAC databases. While DeepAC allows for soft-tissue AC, it may not provide the additional anatomic information available from CTAC, such as calcium. Therefore, some centers will want to carefully weigh the benefits of implementing techniques as disclosed herein to reduce radiation exposure against the added clinical information available from continuing with CTAC imaging. In some cases, the results from a DeepAC analysis can be used to generate a recommendation whether or not to proceed with a CTAC or other CT scan to obtain the additional anatomic information available from traditional AC scans.

As disclosed herein, a DL model was developed which generates DeepAC images from NC reconstructed short-axis slices. DeepAC images provide more similar quantitative assessment of perfusion to actual AC images compared to NC images, which translates into improved diagnostic accuracy for obstructive CAD in external testing. The simulated AC images may simplify the task of artifact identification for physicians compared to NC images alone and can be performed without the need for CTAC hardware.

According to a third example study, a 3D attention gated conditional GAN was trained to generate Pseudo-AC using 798 pairs (48% female) of non-attenuation (NC) corrected and AC. The DL model was evaluated on an external cohort (n=502, 49% male). Change analysis (sum of absolute voxel by voxel count changes) between pairs was used clinically for detection of subtle differences. Positive change accounted for increased counts on AC where defects are corrected, and negative change showed decreased counts (defects are unmasked). Also compare was standard GAN evaluation methods like Wasserstein distance and Frechet inception distance (FID) applied to natural images. Wilcoxon rank sum test was used to compare median values of perfusion change.

In the third example study, the cGAN network was trained using short-axis SPECT AC images (reconstructed at 4×4×4 mm with slice thickness of 4 mm) were used. Mean normalization was used to normalize the NC and AC images sets with the mean value of the NC images. To mitigate potential background artifacts, 32×32×32 volumes of interest centered on the left ventricle were used as the input.

The generator model was an Attention UNet 3D model with instance normalization and discriminator similar to the pix2pix model. The generator includes 4 level 3D UNet along with an attention gate which combines the input from a lower level with the skipped connection and helps the generator to implicitly focus on relevant structures at a lower computational overhead. The discriminator and the generator were trained together like game theory, with no additional benefit to either the generator or the discriminator, so that the game remains in equilibrium. The cost function included standard GAN loss, along with absolute error between the Pseudo-AC and AC. The weights for the proposed architecture were initialized by Xavier initialization and Adam optimizer was used to linearly decay the learning rate to 0. Momentum of 0.5 and an initial learning rate of 0.0002 were set. The model was evaluated on an external cohort (n=502, 49% male).

Change analysis (sum of absolute voxel by voxel count changes) between pairs was used clinically for detection of subtle differences. Quantitive clinical change analysis were performed on Quantitative Perfusion SPECT (QPS) software (Cedars-Sinai Medical Center, Los Angeles, CA).

Standard GAN evaluation methods like Wasserstein distance and Frechet inception distance (FID) (lower is better) were used for natural image evaluation. Wilcoxon rank sum test was used to compare median values of perfusion change as well as FID and Wasserstein distance.

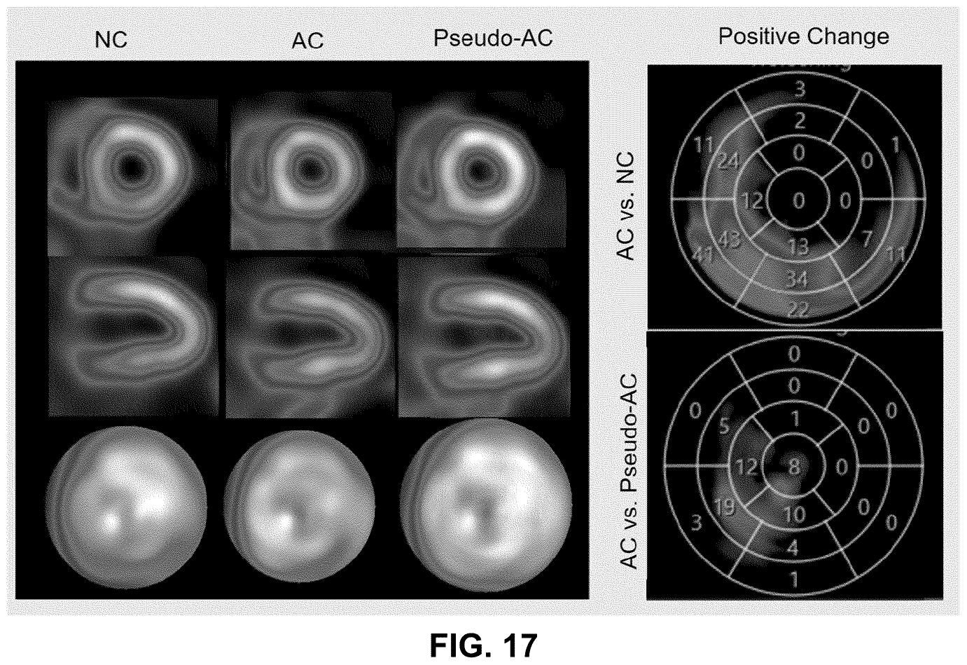

FIG. 17 is a combination image depicting multiple view of different types of SPECT-MPI data and positive change charts for a fourth example patient according to certain aspects of the present disclosure. Positive change accounted for increased counts on AC where defects were corrected, and negative change showed decreased counts with respect to the NC image (defects were unmasked). For the fourth example patient, FID was 149.18 for AC vs Pseudo-AC and 152.29 for NC vs Pseudo-AC. The Wasserstein distance was 393.44 for AC vs Pseudo-AC and 399.08 for NC vs Pseudo-AC.

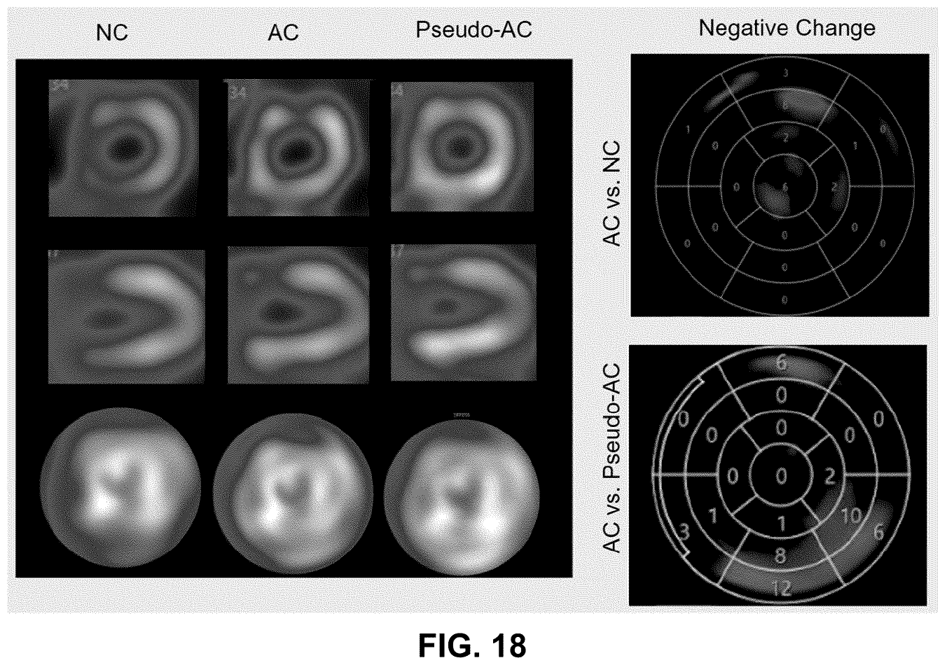

FIG. 18 is a combination image depicting multiple view of different types of SPECT-MPI data and positive change charts for a fifth example patient according to certain aspects of the present disclosure. Negative change highlighted regions where attenuation artifacts could be generated for cases with higher Body Mass Index (BMI). In other words, higher BMI can cause large artifacts which are visible by negative change. Additional clinical insights are hence provided through such change analysis. For the fifth example patient, FID was 156.29 for AC vs Pseudo-AC and 164.13 for NC vs Pseudo-AC. The Wasserstein distance was 528.72 for AC vs Pseudo-AC and 556.40 for NC vs Pseudo-AC.

In this third example study, positive change between AC vs NC (8.6, Inter Quartile Range [IQR] 5.46-12.77, p<0.001) was significantly higher than AC vs Pseudo-AC (median 1.71, [IQR] 0.63, 3.24). Negative change was higher for AC vs NC (median 1.74, (IQR) 0.98-2.97, p<0.001) than for AC vs Pseudo-AC (1.12, [IQR] 0.33-2.24). However, there was non-significant difference for AC vs Pseudo-AC and NC vs Pseudo-AC FID (157.37, 157.97, p=0.09) and Wasserstein distance (402.26, 406.68, p=0.55) respectively. Mean Time for Pseudo-AC generation on central processing unit (CPU) was 66 milliseconds.

Rapid Pseudo-AC generation with quantified change analysis was externally validated. However, standard GAN evaluation techniques suitable for evaluation of natural images did not appear to be as strong of a discriminator for MPI purposes.

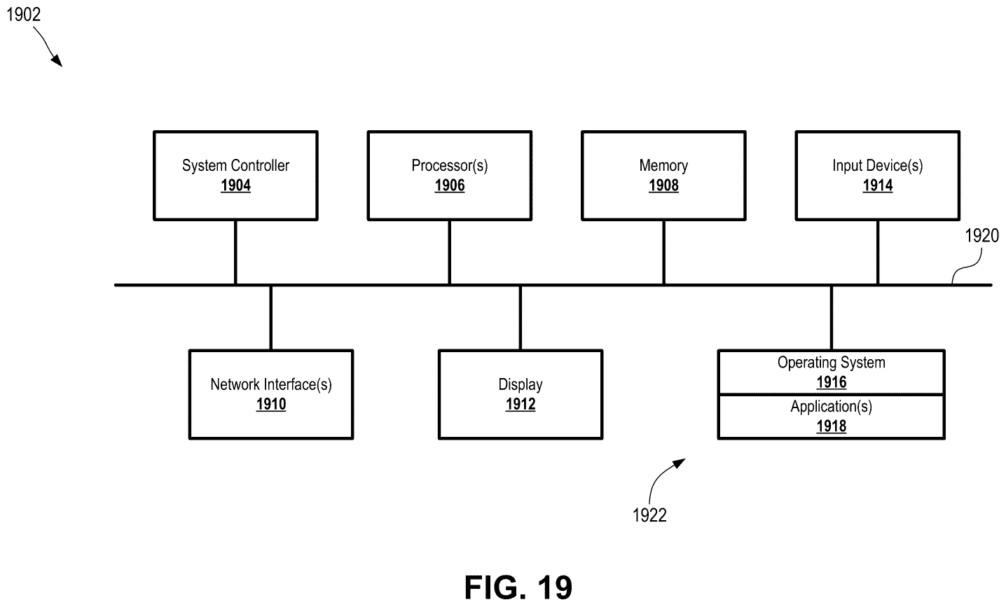

FIG. 19 is a block diagram of an example system architecture 1902 for implementing features and processes of the present disclosure, such as those presented with reference to processes 300 and 400 of FIGS. 3 and 4, respectively. The features and processes disclosed herein can be implemented using one or multiple instances of 1902. The system architecture 1902 can be used to implement a server (e.g., a cloud-accessible server), a user device (e.g., a smartphone or personal computer), or any other suitable device for performing some or all of the aspects of the present disclosure. The system architecture 1902 can be implemented on any electronic device that runs software applications derived from compiled instructions, including without limitation personal computers, servers, smart phones, electronic tablets, game consoles, email devices, and the like. In some implementations, the system architecture 1902 can include one or more processors 1906, one or more input devices 1914, one or more display devices 1912, one or more network interfaces 1910, and one or more computer-readable media 1922. Each of these components can be coupled by bus 1920.

Display device 1912 can be any known display technology, including but not limited to display devices using Liquid Crystal Display (LCD) or Light Emitting Diode (LED) technology. Processor(s) 802 can use any known processor technology, including but not limited to graphics processors and multi-core processors. Input device 1914 can be any known input device technology, including but not limited to a keyboard (including a virtual keyboard), mouse, track ball, and touch-sensitive pad or display. In some cases, audio inputs can be used to provide audio signals, such as audio signals of an individual speaking. Bus 1920 can be any known internal or external bus technology, including but not limited to ISA, EISA, PCI, PCI Express, NuBus, USB, Serial ATA or FireWire.

Computer-readable medium 1922 can be any medium that participates in providing instructions to processor 1906 for execution, including without limitation, non-volatile storage media (e.g., optical disks, magnetic disks, flash drives, etc.) or volatile media (e.g., SDRAM, ROM, etc.). The computer-readable medium (e.g., storage devices, mediums, and memories) can include, for example, a cable or wireless signal containing a bit stream and the like. However, when mentioned, non-transitory computer-readable storage media expressly exclude media such as energy, carrier signals, electromagnetic waves, and signals per se.

Computer-readable medium 1922 can include various instructions for implementing operating system 1916 and applications 1918 such as computer programs. The operating system 1916 can be multi-user, multiprocessing, multitasking, multithreading, real-time and the like. The operating system 1916 performs basic tasks, including but not limited to: recognizing input from input device 1914; sending output to display device 1912; keeping track of files and directories on computer-readable medium 1922; controlling peripheral devices (e.g., storage drives, interface devices, etc.) which can be controlled directly or through an I/O controller; and managing traffic on bus 1920. Computer-readable medium 1922 can include various instructions for implementing firmware processes, such as a BIOS. Computer-readable medium 1922 can include various instructions for implementing any of the processes described herein, including at least processes 300 and 400 of FIGS. 3 and 4, respectively.

Memory 1908 can include high-speed random access memory and/or non-volatile memory, such as one or more magnetic disk storage devices, one or more optical storage devices, and/or flash memory (e.g., NAND, NOR). The memory 1908 (e.g., computer-readable storage devices, mediums, and memories) can include a cable or wireless signal containing a bit stream and the like. However, when mentioned, non-transitory computer-readable storage media expressly exclude media such as energy, carrier signals, electromagnetic waves, and signals per se. The memory 1908 can store an operating system, such as Darwin, RTXC, LINUX, UNIX, OS X, WINDOWS, or an embedded operating system such as VxWorks.

System controller 1904 can be a service processor that operates independently of processor 1906. In some implementations, system controller 1904 can be a baseboard management controller (BMC). For example, a BMC is a specialized service processor that monitors the physical state of a computer, network server, or other hardware device using sensors and communicating with the system administrator through an independent connection. The BMC is configured on the motherboard or main circuit board of the device to be monitored. The sensors of a BMC can measure internal physical variables such as temperature, humidity, power-supply voltage, fan speeds, communications parameters and operating system (OS) functions.

The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language (e.g., Objective-C, Java), including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors or cores, of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer.

The features can be implemented in a computing system that includes a back-end component, such as a data server, or that includes a middleware component, such as an application server or an Internet server, or that includes a front-end component, such as a client computer having a graphical user interface or an Internet browser, or any combination thereof. The components of the system can be connected by any form or medium of digital data communication such as a communication network. Examples of communication networks include, e.g., a LAN, a WAN, and the computers and networks forming the Internet.

The computing system can include clients and servers. A client and server are generally remote from each other and typically interact through a network. The relationship of client and server arises by virtue of computer programs running on the respective computers and having a client-server relationship to each other.

One or more features or steps of the disclosed embodiments can be implemented using an application programming interface (API). An API can define one or more parameters that are passed between a calling application and other software code (e.g., an operating system, library routine, function) that provides a service, that provides data, or that performs an operation or a computation.

The API can be implemented as one or more calls in program code that send or receive one or more parameters through a parameter list or other structure based on a call convention defined in an API specification document. A parameter can be a constant, a key, a data structure, an object, an object class, a variable, a data type, a pointer, an array, a list, or another call. API calls and parameters can be implemented in any programming language. The programming language can define the vocabulary and calling convention that a programmer will employ to access functions supporting the API.

In some implementations, an API call can report to an application the capabilities of a device running the application, such as input capability, output capability, processing capability, power capability, communications capability, and the like.

The foregoing description of the embodiments, including illustrated embodiments, has been presented only for the purpose of illustration and description and is not intended to be exhaustive or limiting to the precise forms disclosed. Numerous modifications, adaptations, and uses thereof will be apparent to those skilled in the art. Numerous changes to the disclosed embodiments can be made in accordance with the disclosure herein, without departing from the spirit or scope of the disclosure. Thus, the breadth and scope of the present disclosure should not be limited by any of the above described embodiments.

Although certain aspects and features of the present disclosure have been illustrated and described with respect to one or more implementations, equivalent alterations and modifications will occur or be known to others skilled in the art upon the reading and understanding of this specification and the annexed drawings. In addition, while a particular feature may have been disclosed with respect to only one of several implementations, such feature may be combined with one or more other features of the other implementations as may be desired and advantageous for any given or particular application.

The terminology used herein is for the purpose of describing particular embodiments only, and is not intended to be limiting of the disclosure. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

One or more elements or aspects or steps, or any portion(s) thereof, from one or more of any of claims 1 to 20 below can be combined with one or more elements or aspects or steps, or any portion(s) thereof, from one or more of any of the other claims 1 to 20 or combinations thereof, to form one or more additional implementations and/or claims of the present disclosure.

What is claimed is:

1. A method, comprising:
receiving non-attenuation-corrected (NC) single photon emission computed tomography (SPECT) imaging data, the NC SPECT imaging data including a plurality of image slices; and
generating simulated attenuation-correction (AC) SPECT imaging data from the NC SPECT imaging data by applying the NC SPECT imaging data to a generator network of a conditional generative adversarial network (cGAN) trained using training data, the training data including a plurality of NC SPECT training images and a corresponding plurality of traditional AC SPECT images.

2. The method of claim 1, wherein the NC SPECT imaging data is associated with a myocardial perfusion imaging (MPI) study, the method further comprising generating a coronary artery disease (CAD) evaluation based at least in part on the attenuation-corrected SPECT imaging data.

3. The method of claim 2, wherein generating the CAD evaluation includes determining a stress total perfusion deficit value based at least in part on the attenuation-corrected SPECT imaging data.

4. The method of claim 3, wherein generating the CAD evaluation includes determining a stress volume value.

5. The method of claim 1, wherein the plurality of NC SPECT training images and the corresponding plurality of traditional AC SPECT images are short-axis SPECT slices.

6. The method of claim 5, wherein each of the short-axis SPECT slices is reconstructed at 4×4×4 mm with a slice thickness of 4 mm.

7. The method of claim 1, wherein the cGAN is trained by supplying as input to the cGAN, for each of the NC SPECT training images and the corresponding traditional AC images, a region of interest centered on a left ventricle within the respective NC SPECT training image.

8. The method of claim 1, wherein the generator network is an Attention UNet 3D model with instance normalization.

9. The method of claim 8, wherein the Attention UNet 3D model includes four levels.

10. The method of claim 19, wherein a cost function of the cGAN includes absolute error between each simulated output and respective ones of the plurality of traditional AC images.

11. The method of claim 1, further comprising presenting the attenuation-corrected SPECT imaging data

12. A computer program product embodied in a non-transitory machine-readable storage medium, comprising instructions which, when executed by a computer, cause the computer to carry out the method of claim 1.

13. A system, comprising:
one or more data processors; and
a non-transitory computer-readable storage medium containing instructions which, when executed on the one or more data processors, cause the one or more data processors to perform operations including:
receiving non-attenuation-corrected (NC) single photon emission computed tomography (SPECT) imaging data, the NC SPECT imaging data including a plurality of image slices; and
generating simulated attenuation-correction (AC) SPECT imaging data from the NC SPECT imaging data by applying the NC SPECT imaging data to a generator network of a conditional generative adversarial network (cGAN) trained using training data, the training data including a plurality of NC SPECT training images and a corresponding plurality of traditional AC SPECT images.

14. The system of claim 13, wherein the NC SPECT imaging data is associated with a myocardial perfusion imaging (MPI) study, the operations further including generating a coronary artery disease (CAD) evaluation based at least in part on the attenuation-corrected SPECT imaging data.

15. The system of claim 14, wherein generating the CAD evaluation includes at least one of i) determining a stress total perfusion deficit value based at least in part on the attenuation-corrected SPECT imaging data; and ii) determining a stress volume value.

16. The system of claim 13, wherein the plurality of NC SPECT training images and the corresponding plurality of traditional AC SPECT images are short-axis SPECT slices.

17. The system of claim 16, wherein each of the short-axis SPECT slices is reconstructed at 4×4×4 mm with a slice thickness of 4 mm.

18. The system of claim 13, wherein the cGAN is trained by supplying as input to the cGAN, for each of the NC SPECT training images and the corresponding traditional AC images, a region of interest centered on a left ventricle within the respective NC SPECT training image.

19. The system of claim 13, wherein the generator network is an Attention UNet 3D model with instance normalization.

20. The system of claim 13, wherein a cost function of the cGAN includes absolute error between each simulated output and respective ones of the plurality of traditional AC images.
